(19) United States
(12) Patent Application Publication
(10) Pub. No.: US 2012/0215064 A1
(43) Pub. Date: Aug. 23, 2012

(54) ENHANCEMENT DEVICE AND METHOD OF MAKING AND USING THE SAME

(76) Inventor: George W. CARROLL, Winter Park, FL (US)

(21) Appl. No.: 13/160,084

(22) Filed: Jun. 14, 2011

Related U.S. Application Data
(63) Continuation of application No. PCT/US2011/025287, filed on Feb. 17, 2011.

(51) Int. Cl. A61F 5/00

(52) U.S. Cl. 600/39

(57) ABSTRACT

An enhancement device includes an elastically expandable member being capable of assuming at least one folded configuration. A first section of the member has a first axial length. A second section of the member has a second axial length and an outer diameter smaller than the first section. Also disclosed is a method of making the device and using the device for deep clitoral stimulation and/or for placing the same on a user's penis as discussed in the instant application.

### CROSS REFERENCE TO RELATED APPLICATION

The instant application is a continuation application of PCT Application No. PCT/US2011/025287 filed on Feb. 17, 2011, designating the United States of America, the disclosure of which, including the specification, drawings and claims, is incorporated herein by reference in its entirety.

### BACKGROUND OF THE INVENTION

1. Field of the Invention

The invention relates to an enhancement device as well as a method of making and using the same. The device can be configured to enhance a sexual experience for the female and can, in particular, be use to provide deep clitoral stimulation. In embodiments, the device may be placed on a penis and/or on a device simulating or being capable of functioning like a penis, e.g., an erect penis.

2. Discussion of Background Information

Enhancement devices for the penis are known, as evidenced by the following U.S. Pat. Nos. which are each hereby expressly incorporated by reference herein in their entireties: U.S. Pat. No. 3,455,301 to CLARK, U.S. Pat. No. 3,621,840 to MACCHIONI, U.S. Pat. No. 4,240,413 to HANUS, U.S. Pat. No. 4,488,541 to GARCIA, U.S. Pat. No. 4,564,006 to POMERANZ, U.S. Pat. No. 4,640,270 to CHIN, U.S. Pat. No. 4,671,262 to WEST, U.S. Pat. No. 5,074,315 to McCUISTON, U.S. Pat. No. 5,314,447 to PAPURT, U.S. Pat. No. 5,868,137 to BROWN, U.S. Pat. No. 6,251,067 to STRICKHOLM, U.S. Pat. No. 6,319,194 to WULF, U.S. Pat. No. 6,436,031 to SALIB, U.S. Pat. No. 6,793,620 to DROZNIN et al., U.S. Pat. No. 7,261,685 to WU, U.S. Pat. No. 7,566,299 to MONTGOMERY, US 2006/0030750 to AMER, US 2006/0278236 to RESNIC, and US 2009/0069629 to USON CALVO. Additional devices can be found in websites such as http://www.calexotics.com/c-313-sleeves.aspx?pagenum=1&sortfield=NEW&sortdirection=ASC&perpage=36

Conventional devices, however, have not been aimed at or specifically configured to mainly enhance a sexual experience for the female. Nor have such devices had as their main aim to provide deep clitoral stimulation. Such devices also lack the advantageous combination of the material and physical configuration utilized in the invention. In particular, many of the conventional devices are designed to fit and be used only at the base of the penis, even though they are configured to help stimulate the female. When such shorter length devices are utilized, only the outer portion of the vagina is targeted for stimulation. Such devices do no take advantage or take into account the recently discovered information regarding deep branches of the clitoris. In contrast, the invention aims to enhance an entire or substantially an entire length of the penis allowing for deep clitoral stimulation as well as for outer stimulation. Another disadvantage or difficulty associated with conventional devices relates to the material of the devices. Many of these devices are made out of a relatively hard material. This may present less of a problem for short length devices, but becomes more of a concern if such sleeves are lengthen. This is because it is more difficult to install or slide-on a longer sleeve onto the penis. It is believed that this is a major reason why many conventional sleeves are relatively short so as to end up being located at the base of the penis. In contrast, the sleeve of the invention can be made longer, i.e., enhance substantially an entire length of the penis, and be made of a material that can be slid-on more easily, i.e., by being made of a material that allows for it to fold onto itself.

Masters and Johnson were the first to determine that the clitoral structures surround and extend along the vagina, determining that all orgasms are of clitoral origin. More recently, Australian urologist Dr. Helen O'Connell using MRI technology noted a direct relationship between the legs or roots of the clitoris and the erectile tissue of the clitoral bulbs and corpora, and the distal urethra and vagina. She asserts that this interconnected relationship is the physiological explanation for the conjectured G-spot and experience of vaginal orgasm taking into account the stimulation of the internal parts of the clitoris during vaginal penetration. The invention aims to take advantage of this new information, further details of which can be found in the following publications: O'Connell, Helen, Anatomy of the Clitoris, J Urol. 2005 October; 174(4 Pt 1):1189-95, PMID 16145367, Federation of Feminist Women's Health Centers (1991). A New View of a Woman's Body. Feminist Heath Press, 46. ISBN 0-929945-0-2, and Mascall, Sharon, “Time for Rethink on the Clitoris”, BBC News. 2006 June |url=http://news.bbc.co.uk/1/hi/health/5013866.stm. The disclosure of each of these publications is hereby expressly incorporated by reference in their entireties.

### SUMMARY OF THE INVENTION

The invention relates to an enhancement device that is specifically and/or mainly configured to enhance a sexual experience for the female and, in particular, to provide deep clitoral stimulation. The device may also be useful in potentially aiding and/or facilitating orgasm in the female.

The invention also relates to a device which comprises an elastically expandable member being capable of assuming at least one folded configuration. A first section of the member has a first axial length. A second section of the member has a second axial length and an outer diameter smaller than the first section.

In embodiments, the first section is sized and configured to be arranged approximately adjacent a base portion of the penis and the second section is sized and configured to be arranged approximately adjacent a head portion of a penis or a member simulating a penis.

In embodiments, the member has open opposite ends and an overall length that is at least 50% or more of an overall length of an erect penis or a member simulating a penis.

In embodiments, the overall length that is at least 60% or more of the overall length of the erect penis or the member simulating a penis.

In embodiments, the overall length that is at least 75% or more of the overall length of the erect penis or the member simulating a penis.

In embodiments, the member has open opposite ends and an overall length that is between about 3.5 inches and about 5.5 inches.

In embodiments, the overall length is about 4.5 inches.

In embodiments, the member is non-inflatable and/or has an axial length that is greater than a largest outside diameter of the member in a resting or non-expanded state.

In embodiments, the member has is sized and configured to exert a compressive force on an erect penis of member simulating an erect penis.

In embodiments, the member has an internal diameter sized and configured to exert a compressive force on an erect penis of member simulating an erect penis at least in an area of the first and second sections.

In embodiments, the member has an internal diameter sized and configured to exert a compressive force on an erect penis or a member simulating an erect penis substantially over an overall axial length of the member. In embodiments, the member may be self-supporting in an axial direction, i.e., when one rests a larger end of the member on a surface, its length will not change under its own weight and/or under the force of gravity. In embodiments, the member may have torsional elasticity and/or may resist torsion and/or may automatically or naturally return to an original state when no longer subjected to a torsion force.

In embodiments, the member completely surrounds a substantial portion of a penis or a substantial portion of a member simulating a penis. The member may have no through openings in a wall of the member which extend between an outside surface and an inside surface of the member.

In embodiments, at least one of the following may be utilized: the member is a one-piece member, the member is substantially transparent or translucent, and the member is generally sleeve-shaped and the first axial length is at least twice as great as the second axial length.

In embodiments, the first and second sections are each generally cylindrical and the first axial length is greater than the second axial length.

In embodiments, at least one of a wall thickness of the first section is approximately ¼ inches and a wall thickness of the second section is approximately ⅛ inches.

In embodiments, the first and second sections have a same internal diameter and/or at least one of the first and second sections may have a substantially smooth outer surface.

In embodiments, the device further comprises at least one of an internal generally cylindrical surface arranged within the member and/or extending over substantially an entire length of the member, oppositely arranged annular surfaces, and/or oppositely arranged concentric generally circular open ends.

In embodiments, at least one of; the member has open opposite ends and at least one third section axially disposed between the first and second sections, and the member has open opposite ends and a third section having a diameter different from the first and second sections and being axially disposed between the first and second sections.

In embodiments, the member has open opposite ends and third and fourth sections having different diameters and axially disposed between the first and second sections.

In embodiments, at least one of the at least one folded configuration is one wherein the first section can be folded back at least partially over the second section such that the second section is arranged substantially axially inside or within the first section, and the at least one folded configuration is one wherein the first section can be folded back at least partially over the second section during installation of the device on an erect penis or a member simulating a penis.

In embodiments, at least one of the at least one folded configuration is one wherein the member can assume both a substantially flattened configuration and a generally cylindrical configuration, the at least one folded configuration is one wherein the member can assume a substantially flattened configuration, the at least one folded configuration is one wherein the member can assume a substantially flattened configuration such that opposite internal surfaces of the one-piece member substantially contact one another, and the at least one folded configuration is one wherein the member can assume a substantially flattened configuration when squeezed between a user's fingers of one hand.

In embodiments, the invention relates to a method of making the device of the type(s) described above comprising forming the member having different diameter first and second sections.

In embodiments, the invention relates to a method of using the device of the type(s) described above comprising installing the member on a penis or a member simulating the penis and inserting the penis or member simulating the penis into a vagina in a manner which results in at least the first section providing stimulation to at least a portion of the vagina.

The invention also relates to an enhancement device comprising an elastically expandable member having open opposite ends and being capable of assuming each of a generally cylindrical shape and at least one folded configuration. A first section of the member has a first axial length. A second section of the member has a second axial length and an outer diameter smaller than the first section. An overall length of the member is at least 50% or more of an overall length of an erect penis. The at least one folded configuration is one wherein the first section can be folded back at least partially over the second section during installation of the device.

The invention also relates to a male or penis enhancement device comprising an elastically expandable one-piece or integrally formed member having open opposite ends and being capable of assuming each of a generally cylindrical configuration and at least one folded configuration. A first section of the one-piece member has a first axial length. A second section of the one-piece member has a second axial length and an outer diameter smaller than the first section. An overall length of the member is at least 50% or more of an overall length of an erect penis. The at least one folded configuration is one wherein the first section can be folded back at least partially over the second section during installation of the device.

The invention also relates to an enhancement device of the type described above and further including anyone, more than one, and/or all of the above-noted features and/or any combination of such features.

The invention also relates to a device for providing deep clitoral stimulation, comprising a member having open opposite ends, a first section of the member having a first axial length, a second section of the member having a second axial length and an outer diameter smaller than the first section, and an overall length that is at least 50% or more of an overall length of an erect penis or a member simulating a penis.

In embodiments, one of the open ends is configured to be arranged behind a head of the penis so that the penis head is exposed and can experience stimulation during stimulation of the female.

In embodiments, at least one transition area is provided between the first and second sections.

In embodiments, at least one short transition area is provided between the first and second sections. In embodiments, the short transition can have an axial length that is less than 25% of an axial length of one or more of the first or section sections. In embodiments, the short transition can have an axial length that is less than 10% of an axial length of one or more of the first or section sections.

In embodiments, at least one long transition area is provided between the first and second sections. In embodiments, the long transition can have an axial length that is more than 25% of an axial length of one or more of the first or section sections. In embodiments, the long transition can have an axial length that is up to 50% of an axial length of one or more of the first or section sections.

Other exemplary embodiments and advantages of the present invention may be ascertained by reviewing the present disclosure and the accompanying drawing.

### BRIEF DESCRIPTION OF THE DRAWINGS

The present invention is further described in the detailed description which follows, in reference to the noted plurality of drawings by way of non-limiting examples of exemplary embodiments of the present invention, in which like reference numerals represent similar parts throughout the several views of the drawings, and wherein:

FIG. 1 shows a side view of a first non-limiting embodiment of the enhancement member. The member is generally sleeve-shaped and utilizes three distinct generally cylindrical sections having three different outer diameters. Two relatively short transitions, e.g., chamfered sections, are utilized;

FIG. 2 shows a side cross-section view of FIG. 1;

FIG. 3 shows a side cross-section view of the device of FIG. 1 in an expanded state;

FIG. 4 shows a rear end view of the device of FIG. 3 in the expanded state illustrating how the member is capable of radial expansion (indicated by arrows);

FIG. 5 shows a side cross-section view of the device of FIG. 1 in a substantially flattened state, i.e., in one of the folded configurations of which the member is capable of assuming;

FIG. 6 shows a rear end view of the device of FIG. 5;

FIG. 7 shows a side cross-section view of the device of FIG. 1 in a substantially folded back state, i.e., in another of the folded configurations of which the member is capable of assuming;

FIG. 8 shows the device of FIG. 7 before being installed on a penis—which is schematically shown in the right side;

FIG. 9 shows the device of FIG. 7 partially installed on a penis. This would typically occur after a lubricant is applied to either the inside of the device or to the outside of the penis (or both) to facilitate installation;

FIG. 10 shows the device of FIG. 7 fully installed on a penis. This occurs after the folded-over portion is folded back towards a base portion of the penis. The fully installed position is, in embodiments, one wherein one end of the device is located at a short but comfortable position behind the head portion of the penis and another end of the device is located at a short but comfortable position in front of the base portion of the penis;

FIG. 11 shows a side view of a second non-limiting embodiment of the enhancement member. The member is generally sleeve-shaped and utilizes four distinct generally cylindrical sections having four different outer diameters. Two relatively short transitions, e.g., chamfered sections, are utilized;

FIG. 12 shows a side cross-section view of FIG. 11;

FIG. 13 shows a side view of the device of FIG. 11 and including axial length dimensions;

FIG. 14 shows a side view of the device of FIG. 1 and including axial length dimensions;

FIG. 15 shows a side view of a third non-limiting embodiment of the enhancement member and including axial length dimensions. The member is generally sleeve-shaped and utilizes two distinct generally cylindrical sections having two different outer diameters. A relatively large transition, e.g., tapered or chamfered section, is arranged between these sections; and

FIG. 16 shows a side cross-section view of FIG. 15.

### DETAILED DESCRIPTION OF THE INVENTION

The particulars shown herein are by way of example and for purposes of illustrative discussion of the embodiments of the present invention only and are presented in the cause of providing, without wishing to be bound by theory, what is believed to be the most useful and readily understood description of the principles and conceptual aspects of the present invention. In this regard, no attempt is made to show structural details of the present invention in more detail than is necessary for the fundamental understanding of the present invention, the description is taken with the drawings making apparent to those skilled in the art how the forms of the present invention may be embodied in practice.

The following is a description of non-limiting embodiments of a device, system and/or method for enhancing the sexual experience of a female by stimulating a specific area of the vagina. In particular, the device is configured to provide deep clitoral stimulation. The device is installable on a penis or on a member simulating a penis which is then inserted into the vagina. Repeated movement of an enlarged section of the device located not only in an area of the base of the penis but also mostly on the entire length of the penis shaft, and the resulting stimulating contact between the same and the vagina, can result (all things being equal) in female reaching orgasm in a shorter time than without using the device and/or can result in more intense orgasms and specifically provides for deep clitoral stimulation. The properties of the material of the device are such as to make the installation on the penis, the use on the penis, and/or the stimulation to the vagina easier, more comfortable, and/or ergonomic than known devices.

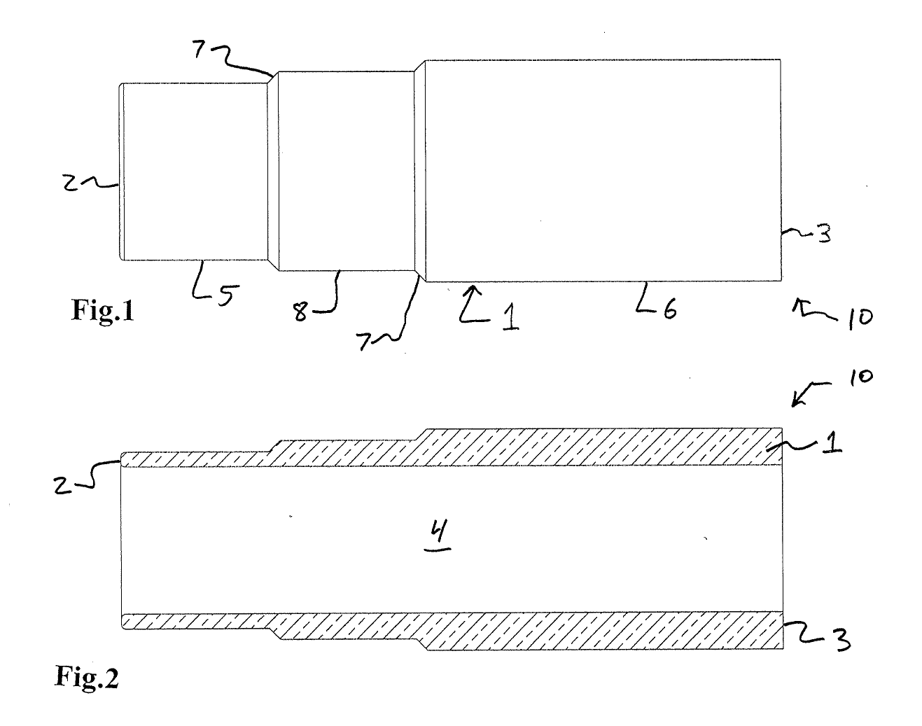

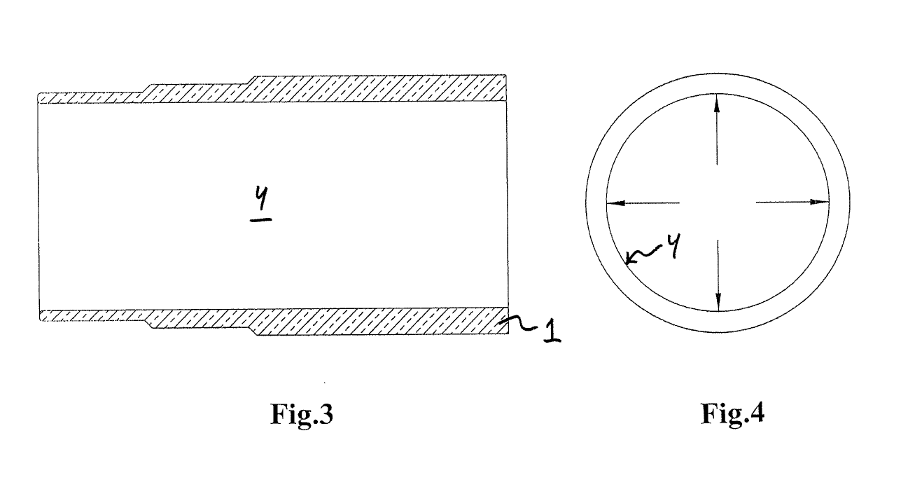

FIGS. 1-7 show one non-limiting embodiment of the invention. The device 10 is essentially comprised of a member 1. The member 1 has first end 2 which is sized and configured to be disposed adjacent a head portion of a penis and a second end 3 which is sized and configured to be disposed adjacent a base portion of a penis. In embodiments, the first end 2 is disposed adjacent a head portion of a penis, but spaced therefrom by a space ranging from about ⅛ inch to about 1 inch, and preferably between about ¼ inch and ½ inch, and the second end 3 is disposed adjacent a base portion of a penis, but spaced therefrom by a space ranging from about ⅛ inch to about 1 inch, and preferably between about ¼ inch and ½ inch. In embodiments, the first end 2 is generally annular and is slightly rounded where it meets the inner surface 4 and where it meets outer surface 5. In embodiments, the second end 3 is generally annular and need not be rounded where in meets the inner surface 4 and where it meets outer surface 6.

In embodiments, the member 1 is a one-piece or integrally formed member. In embodiments, the member 1 can be formed from any suitable material, e.g., a synthetic resin (e.g., plastic or synthetic rubber). In embodiments, the material can be a medical grade and/or a flexible and/or semi-rigid synthetic resin material. In embodiments, the member 1 is made of a transparent or translucent material. In embodiments, the member 1 is made of a material from the family of thermoplastic elastomers. In embodiments, the member 1 is made of a silicone material or silicone/rubber mixtures. One non-limiting advantageous material for the member 1 is Dermasol DS-303 made by or obtainable from the company California Medical Innovations of Pomona, Calif. In embodiments, the member 1 is non-porous and/or does not absorb fluid and/or is made of a solid material. In embodiments, the member 1 is made of an elastically expandable material and/or capable of being of assuming at least one folded configuration as will be described below. In embodiments, the member 1 has the configuration shown in the drawings when made, has an amount of rigidity which allows it to assume the configuration shown in the drawings when a cylindrical shaft is inserted within the member 1 and having an outside diameter which matches that of the inside diameter of the main axial opening, but does not maintain that same shape when the shaft is removed and the member 1 is placed on its side (as shown in FIG. 1), i.e., the member 1 will experience some deflection due to the force of gravity and its own weight.

The member 1 also has a generally or substantially cylindrical inner surface 4 which defines a main axial through opening and extends along substantially the entire length of the member 1. This inner surface 4 is sized and configured to be disposed over a shaft portion of a penis and to exert a compressive force thereon so as to create at least a snug fit. This also prevents, reduces or minimizes axial or rotational movement of the member 1 relative to the penis.

The member 1 further also has plural generally or substantially cylindrical outer surfaces 5, 6 and 8 which define distinct and separate sections of the member 1. Section 5 is sized and configured to be disposed adjacent a head portion of a penis, has a uniform but thinner wall thickness, and a predetermined axial length. In embodiments, a wall thickness of section 5 is between about 0.05 inch and about 0.15 inch, and can preferably be about ⅛ inch. Section 6 is sized and configured to be disposed adjacent a base portion of a penis, has a uniform but thicker wall thickness, and a predetermined axial length. In embodiments, the axial length of section 6 is greater than that of section 5. In embodiments, a wall thickness of section 6 is between about 0.2 inch and about 0.3 inch, and can preferably be about ¼ inch. Section 8 is sized and configured to be disposed intermediate the base and head portions of a penis, has a uniform but intermediate wall thickness, and a predetermined axial length. In embodiments, the axial length of section 8 is less than that of section 6. In embodiments, a wall thickness of section 8 is between about 0.15 inch and about 0.25 inch, and can preferably be about 3/16 inch.

The member 1 further also has plural generally or substantially tapered or chamfered outer surfaces 7 which define distinct and separate short transition sections of the member 1. Each section 7 is sized and configured to provide a short but smooth and comfortable transition between sections 5 and 8 as well as between sections 8 and 6. In embodiments, an axial length of each section 7 is between about 0.05 inch and about 0.15 inch, and can preferably be about ⅛ inch.

As shown in FIGS. 3 and 4, the member 1 can be capable of radial expansion (indicated by arrows) in embodiments. In embodiments, the member 1 is capable of assuming an original state after the force(s) causing radial expansion are removed, i.e., the member 1 has memory which allows it to expand elastically and thereafter return to a relaxed state which is the same or nearly the same as an original resting state. This elastic expansion allows the member 1 to be more easily installed on a penis or a member simulating the penis.

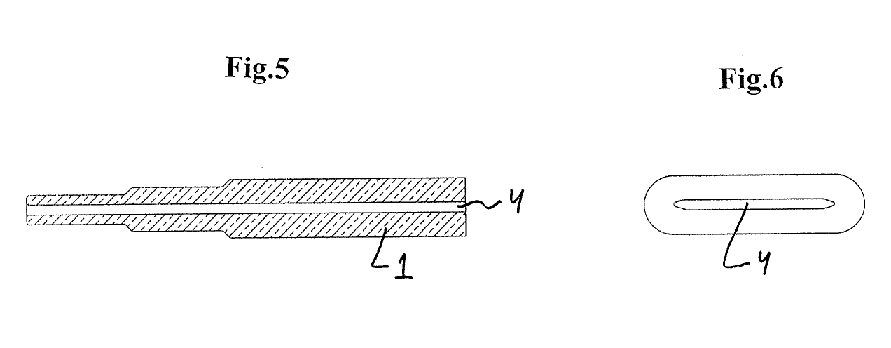

As shown in FIGS. 5 and 6, the member 1 can be capable of being flattened in embodiments. In embodiments, the member 1 is capable of assuming an original state after the force(s) causing the flattening shown in FIGS. 5 and 6 are removed, i.e., the member 1 has memory which allows it to deflect elastically and thereafter return to a relaxed state which is the same or nearly the same as an original resting state. This flattening allows the member 1 to be more easily packaged.

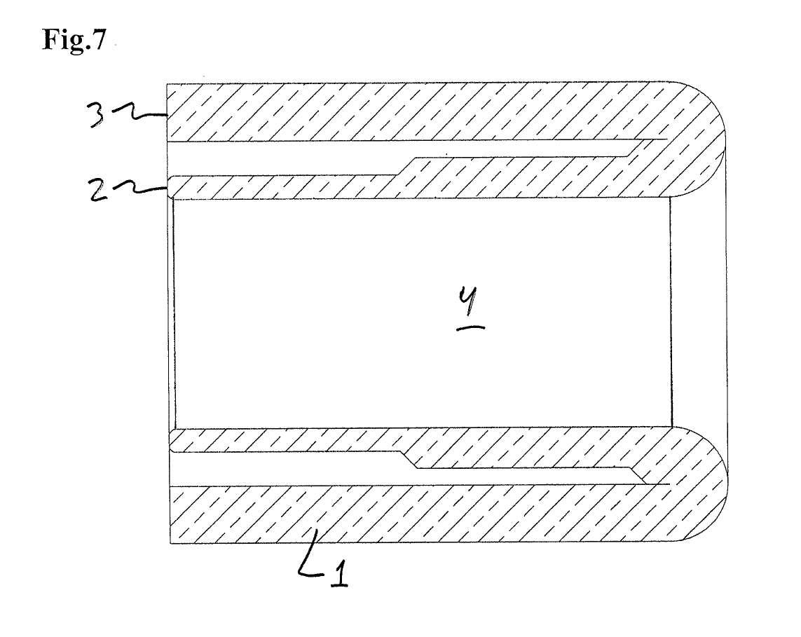

As shown in FIG. 7, the member 1 can be capable of being folded over in embodiments. In embodiments, the member 1 is capable of assuming an original state after the member 1 is folded as shown in FIG. 7, i.e., the member 1 has memory which allows it to deflect and bend elastically and thereafter return to a relaxed state which is the same or nearly the same as an original resting state. This allows the member 1 to be more easily installed on a penis or a member simulating the penis.

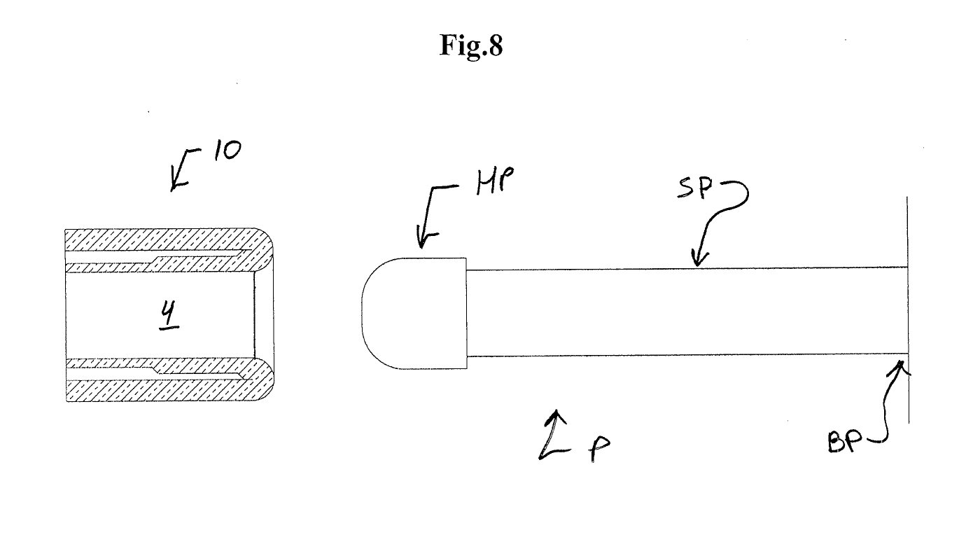

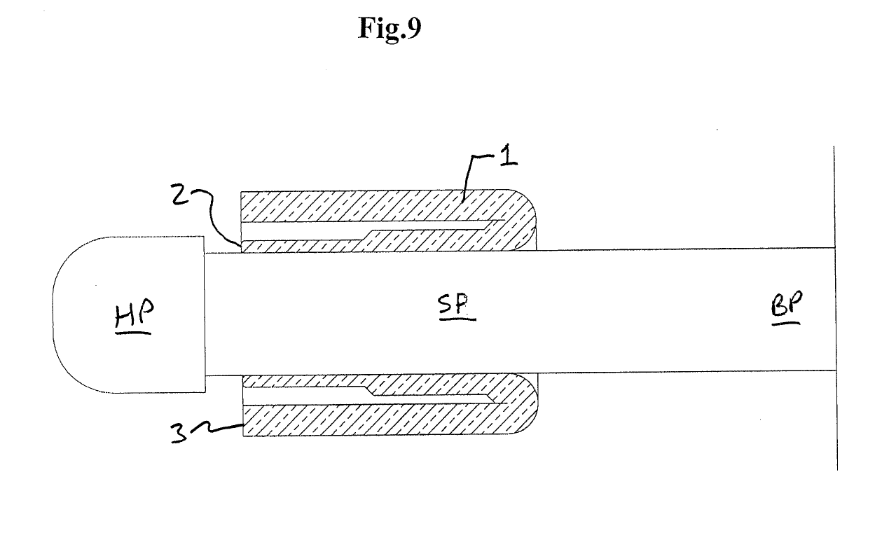

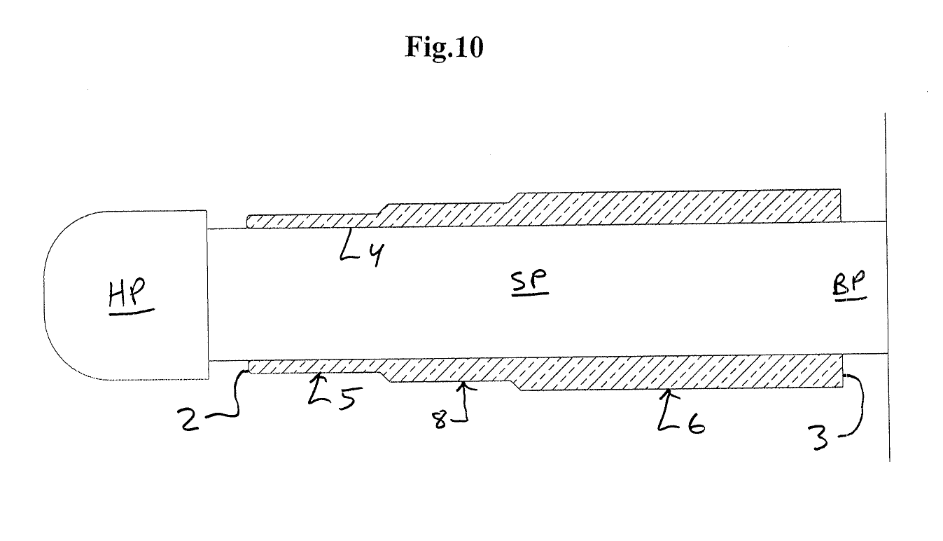

FIGS. 8-10 show one non-limiting way in which the member 1 of FIGS. 1-7 can be installed on a penis. In FIG. 8, the member 1 is shown in a folded back configuration and is ready to be installed on a penis P—which is schematically shown in the right side. The penis P has a head portion HP, a shaft portion SP and a base portion BP. FIG. 9 shows the member 1 partially installed the penis P. This would typically occur after a lubricant is applied to either the inside of the member 1, i.e., on surface 4, or to the outside of the penis P (or both) to facilitate installation. In embodiments, the lubricant is a thin fluid type lubricant such as a commercially available personal lubricant, e.g., astroglide. Although not shown, the penis P can have, in embodiments, a condom (not shown) already installed thereon before the member 1 is placed on the penis P in the manner shown in FIG. 9. As is shown in FIG. 9, the first end 2 of the member 1 is can be placed adjacent to and slightly spaced from the head portion HP of the penis P. FIG. 10 shows the member 1 fully installed on the penis P. This occurs after the folded-over portion of the member 1 is folded back towards a base portion BP of the penis P. The fully installed position is, in embodiments, one wherein the first end 2 of the member 1 is located at a short but comfortable position behind the head portion HP of the penis P and the second end 3 of the member 1 is located at a short but comfortable position in front of the base portion BP of the penis P.

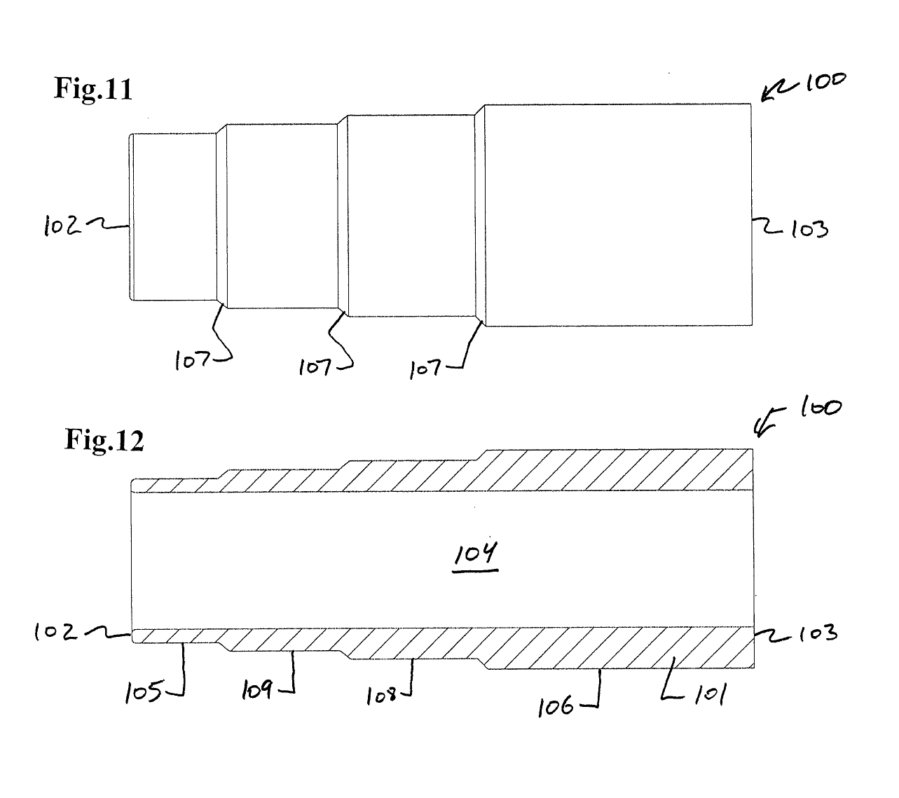

FIGS. 11 and 12 show another non-limiting embodiment of the invention. As in the previous embodiment, the device 100 is essentially comprised of a member 101. The member 101 has first end 102 which is sized and configured to be disposed adjacent a head portion of a penis and a second end 103 which is sized and configured to be disposed adjacent a base portion of a penis. In embodiments, the first end 102 is disposed adjacent a head portion of a penis, but spaced therefrom by a space ranging from about ⅛ inch to about 1 inch, and preferably between about ¼ inch and ½ inch, and the second end 103 is disposed adjacent a base portion of a penis, but spaced therefrom by a space ranging from about ⅛ inch to about 1 inch, and preferably between about ¼ inch and ½ inch. In embodiments, the first end 102 is generally annular and is slightly rounded where in meets the inner surface 104 and where it meets outer surface 105. In embodiments, the second end 103 is generally annular and need not be rounded where in meets the inner surface 104 and where it meets outer surface 106.

In embodiments, the member 101 is a one-piece or integrally formed member. In embodiments, the member 101 is made of any of the materials used in the previously described embodiment. In embodiments, the member 101 is made of an elastically expandable material and/or capable of being of assuming at least one folded configuration as will be described below. In embodiments, the member 101 has the configuration shown in the drawings when made, has an amount of rigidity which allows it to assume the configuration shown in the drawings when a cylindrical shaft is inserted within the member 101 and having an outside diameter which matches that of the inside diameter of the main axial opening, but does not maintain that same shape when the shaft is removed and the member 101 is placed on its side (as shown in FIG. 11), i.e., the member 1 will experience some deflection due to the force of gravity and its own weight.

The member 101 also has a generally or substantially cylindrical inner surface 104 which defines a main axial through opening and extends along substantially the entire length of the member 104. This inner surface 104 is sized and configured to be disposed over a shaft portion of a penis and to exert a compressive force thereon so as to create at least a snug fit. This also prevents, reduces or minimizes axial or rotational movement of the member 1 relative to the penis.

The member 101 further also has plural generally or substantially cylindrical outer surfaces 105, 106, 108 and 109 which define distinct and separate sections of the member 1. Section 105 is sized and configured to be disposed adjacent a head portion of a penis, has a uniform but thinner wall thickness, and a predetermined axial length. In embodiments, a wall thickness of section 105 is between about 0.05 inch and about 0.15 inch, and can preferably be about ⅛ inch. Section 106 is sized and configured to be disposed adjacent a base portion of a penis, has a uniform but thicker wall thickness, and a predetermined axial length. In embodiments, the axial length of section 106 is greater than that of section 105. In embodiments, a wall thickness of section 106 is between about 0.2 inch and about 0.3 inch, and can preferably be about ¼ inch. Section 108 is sized and configured to be disposed intermediate the base and head portions of a penis, has a uniform but intermediate wall thickness, and a predetermined axial length. In embodiments, the axial length of section 108 is less than that of section 106. In embodiments, a wall thickness of section 108 can preferably be about 7/32 inch. Section 109 is also sized and configured to be disposed intermediate the base and head portions of a penis, has a uniform but intermediate wall thickness, and a predetermined axial length. In embodiments, the axial length of section 109 is less than that of section 106. In embodiments, a wall thickness of section 109 can preferably be about 3/16 inch.

The member 101 further also has plural generally or substantially tapered or chamfered outer surfaces 107 which define distinct and separate short transition sections of the member 101. Each section 107 is sized and configured to provide a short but smooth and comfortable transition between sections 105 and 109, between sections 109 and 108, as well as between sections 108 and 106. In embodiments, an axial length of each section 107 is between about 0.05 inch and about 0.15 inch, and can preferably be about ⅛ inch.

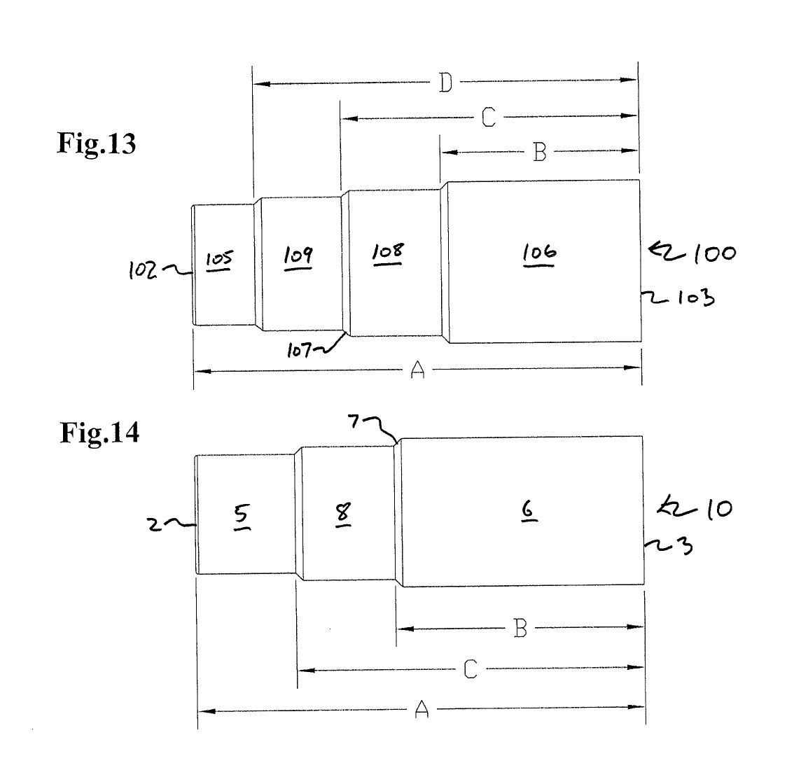

FIG. 13 shows a side view of the device of FIG. 11 and including axial length dimensions in accordance with one non-limiting embodiment of the invention. Thus, the diameter of section 105 can be the result of about a 1 inch inside diameter plus a wall thickness of about 0.1 inches for an outside diameter of 1.2 inches. The overall axial length A of member 100 can be between about 4.5 inches. The axial length A minus D of section 105 can be between about 1 inch. The diameter of section 109 can be the result of about a 1 inch inside diameter plus a wall thickness of about 0.15 inches for an outside diameter of 1.3 inches. The axial length D minus C of section 109 can be between about 1 inch. The diameter of section 108 can be the result of about a 1 inch inside diameter plus a wall thickness of about 0.2 inches for an outside diameter of 1.4 inches. The axial length C minus B of section 108 can be between about 1 inch. The diameter of section 106 can be the result of about a 1 inch inside diameter plus a wall thickness of about 0.25 inches for an outside diameter of 1.5 inches. The axial length B of section 106 can be between about 1.5 inch. In embodiments, each of sections 105, 106, 108, and 109 have the same axial length, e.g., 1.125 inches when the dimension A is 4.5 inches. In embodiments, each of sections 105, 109, and 108 have the same axial length, e.g., 1 inches, and section 106 has an axial length equal to two-times an axial length of section 105 when the dimension A is 4.5 inches. This later embodiment may utilize a wall thickness of 0.25 for section 108 and 0.375 for section 106. In embodiments, sections 105, 109, 108 and 106 have the same axial length, e.g., 1 inches, when the dimension A is 4 inches. This later embodiment may utilize a wall thickness of 0.125 for section 105, 0.2 for section 109, 0.3 for section 108, and 0.375 for section 106.

FIG. 14 shows a side view of the device of FIG. 1 and including axial length dimensions in accordance with one non-limiting embodiment of the invention. Thus, the diameter of section 5 can be the result of about a 1 inch inside diameter plus a wall thickness of about 0.1 inches for an outside diameter of 1.2 inches. The overall axial length A of member 10 can be between about 4.5 inches. The axial length A minus C of section 5 can be between about 1 inch. The diameter of section 8 can be the result of about a 1 inch inside diameter plus a wall thickness of about 0.25 inches for an outside diameter of 1.5 inches. The axial length C minus B of section 8 can be about 1 inch. The diameter of section 6 can be the result of about a 1 inch inside diameter plus a wall thickness of about 0.375 inches for an outside diameter of 1.75 inches. The axial length B of section 6 can be about 2.5 inch. In embodiments, each of sections 5 and 8 have the same axial length, e.g., 1 inch, when the dimension A is 4.5 inches. In embodiments, sections 5 and 8 have the same axial length, e.g., 1 inches, and section 6 has an axial length equal to two-times an axial length of section 5 when the dimension A is 4 inches. This later embodiment may utilize a wall thickness of 0.25 inches for section 5, 0.3 inches for section 8, and 0.375 inches for section 6, when the inside diameter of the sleeve 10 is 0.875 inches. In embodiments, sections 5 and 8 have the same axial length, e.g., 1 inches, and section 6 has an axial length equal to an axial length of both sections 5 and 6 when the dimension A is 4 inches. This later embodiment may utilize a wall thickness of 0.25 inches for section 5, 0.3 inches for section 8, and 0.375 inches for section 6, when the inside diameter of the sleeve 10 is 0.75 inches.

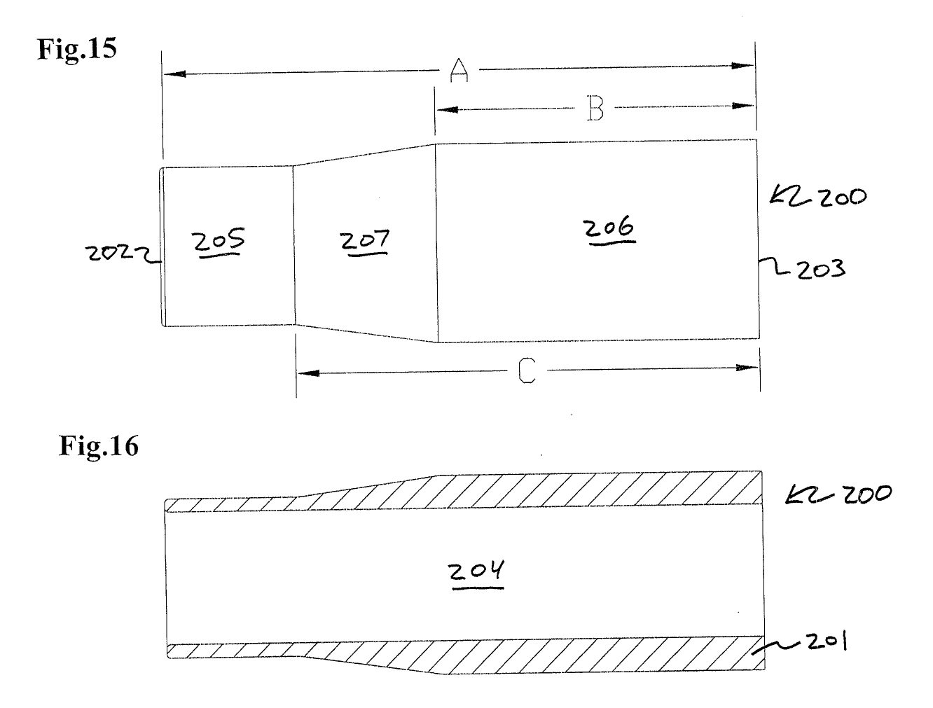

FIGS. 15 and 16 show still another non-limiting embodiment of the invention. The device 200 is essentially comprised of a member 201. The member 201 has first end 202 which is sized and configured to be disposed adjacent a head portion of a penis and a second end 203 which is sized and configured to be disposed adjacent a base portion of a penis. In embodiments, the first end 202 is disposed adjacent a head portion of a penis, but spaced therefrom by a space ranging from about ⅛ inch to about 1 inch, and preferably between about ¼ inch and ½ inch, and the second end 203 is disposed adjacent a base portion of a penis, but spaced therefrom by a space ranging from about ⅛ inch to about 1 inch, and preferably between about ¼ inch and ½ inch. In embodiments, the first end 202 is generally annular and is slightly rounded where in meets the inner surface 204 and where it meets outer surface 205. In embodiments, the second end 203 is generally annular and need not be rounded where in meets the inner surface 204 and where it meets outer surface 206.

In embodiments, the member 201 is a one-piece or integrally formed member. In embodiments, the member 201 is made of any of the materials used in the previous embodiments. In embodiments, the member 201 is made of an elastically expandable material and/or capable of being of assuming at least one folded configuration as will be described below. In embodiments, the member 201 has the configuration shown in the drawings when made, has an amount of rigidity which allows it to assume the configuration shown in the drawings when a cylindrical shaft is inserted within the member 201 and having an outside diameter which matches that of the inside diameter of the main axial opening, but does not maintain that same shape when the shaft is removed and the member 201 is placed on its side (as shown in FIG. 15), i.e., the member 201 will experience some deflection due to the force of gravity and its own weight.

The member 201 also has a generally or substantially cylindrical inner surface 204 which defines a main axial through opening and extends along substantially the entire length of the member 201. This inner surface 204 is sized and configured to be disposed over a shaft portion of a penis and to exert a compressive force thereon so as to create at least a snug fit. This also prevents, reduces or minimizes axial or rotational movement of the member 201 relative to the penis.

The member 201 further also has plural generally or substantially cylindrical outer surfaces 205 and 206 which define distinct and separate sections of the member 201. Section 205 is sized and configured to be disposed adjacent a head portion of a penis, has a uniform but thinner wall thickness, and a predetermined axial length. In embodiments, a wall thickness of section 205 is between about 0.05 inch and about 0.15 inch, and can preferably be about ⅛ inch. Section 206 is sized and configured to be disposed adjacent a base portion of a penis, has a uniform but thicker wall thickness, and a predetermined axial length. In embodiments, the axial length of section 206 is greater than that of section 205. In embodiments, a wall thickness of section 206 is between about 0.2 inch and about 0.3 inch, and can preferably be about ¼ inch.

The member 201 further also has a substantially tapered or chamfered outer surface 207 which defines a distinct and separate long transition section of the member 201. Section 207 is sized and configured to provide a long, smooth and comfortable transition between sections 205 and 206. In embodiments, an axial length of section 207 is between about ¾ inch and about 1¼ inch, and can preferably be about 1 inch.

FIG. 15 also shows axial length dimensions in accordance with one non-limiting embodiment of the invention. Thus, the diameter of section 205 can be between about 1 inch and about 1.25 inch. The diameter of section 206 can be between about 1.5 inch and about 1.75 inch. The overall axial length A of member 201 can be between about 4 inches and about 5 inch, and can preferably be about 4 or 4.5 inches. The axial length B of member 201 can be between about half of length A. The axial length C of member 201 can be between about twice the axial length of section 205.

The invention further contemplates, in regards to each of the herein disclosed embodiments, utilizing additional features, accessories or functions on the sleeve such as vibration (see U.S. Pat. No. 5,377,692 to PFEIL, U.S. Pat. No. 6,485,408 to ORTEN, US 2006/0030749 to HUNG et al., US 2006/0178602 to TENG et al., US 2007/0051373 to LIN, US 2009/0012355 to LIN), electronics (see US 2010/0043803 to OSTERBERG, US 2004/0133070 to LIN), pneumatics (see US 2006/0229494 to WU), and illumination (see U.S. Pat. No. 6,991,600 to WANG, US 2007/0179337 to KALVATANOND et al.). The disclosure of each of these US patent/patent application documents is hereby expressly incorporated by reference in their entireties.

It is noted that the foregoing examples have been provided merely for the purpose of explanation and are in no way to be construed as limiting of the present invention. While the present invention has been described with reference to exemplary embodiments, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. An enhancement device comprising:
an elastically expandable member being capable of assuming at least one folded configuration;
a first section of the member having a first axial length; and
a second section of the member having a second axial length and an outer diameter smaller than the first section,
wherein, in an installed state, the elastically expandable member is not folded upon itself, and
wherein the first section is sized and configured to be arranged approximately adjacent a base portion of a penis or member simulating the penis and the second section is sized and configured to be arranged approximately adjacent a head portion of the penis or member simulating the penis.

2. The device of claim 1, wherein the member has open opposite ends and an overall length, measured when the member is in a pre-installed and non-folded state, that is at least 50% or more of an overall length of an erect penis or member simulating a penis.

3. The device of claim 2, wherein the overall length that is at least 60% or more of the overall length of the erect penis or the member simulating a penis.

4. The device of claim 3, wherein the overall length that is at least 75% or more of the overall length of the penis when erect.

5. The device of claim 1, wherein the member has open opposite ends and an overall length, measured when the member is in a pre-installed and non-folded state, that is between about 3.5 inches and about 5.5 inches.

6. The device of claim 5, wherein the overall length is about 4.5 inches.

7. The device of claim 1, wherein at least one of:
the member is a one-piece member;
the member is substantially transparent and/or translucent;
the member is generally sleeve-shaped and the first axial length is at least twice as great as the second axial length;
the member is non-inflatable;
the member completely surrounds a substantial portion of a penis or a substantial portion of a member simulating a penis;
the member has is sized and configured to exert a compressive force on an erect penis of member simulating an erect penis;
the member has an internal diameter sized and configured to exert a compressive force on an erect penis of member simulating an erect penis at least in an area of the first and second sections; and
the member has an internal diameter sized and configured to exert a compressive force on an erect penis of member simulating an erect penis substantially over an overall axial length of the member.

8. The device of claim 1, wherein the first and second sections are each generally cylindrical and the first axial length is greater than the second axial length.

9. The device of claim 1, wherein at least one of:
a wall thickness of the first section is approximately ¼ inches; and
a wall thickness of the second section is approximately ⅛ inches.

10. The device of claim 1, wherein the first and second sections have a same internal diameter.

11. The device of claim 1, further comprising an internal generally cylindrical surface arranged within the member.

12. The device of claim 1, wherein at least one of:
the member has open opposite ends and at least one third section axially disposed between the first and second sections; and
the member has open opposite ends and a third section having a diameter different from the first and second sections and being axially disposed between the first and second sections.

13. The device of claim 1, wherein the member has open opposite ends and third and fourth sections having different diameters and axially disposed between the first and second sections.

14. The device of claim 1, wherein at least one of:
the at least one folded configuration is one wherein the first section can be folded back at least partially over the second section such that the second section is arranged substantially axially inside or within the first section; and
the at least one folded configuration is one wherein the first section can be folded back at least partially over the second section during installation of the device on an erect penis.

15. The device of claim 1, wherein at least one of:
the at least one folded configuration is one wherein the member can assume both a substantially flattened configuration and a generally cylindrical configuration;
the at least one folded configuration is one wherein the member can assume a substantially flattened configuration;
the at least one folded configuration is one wherein the member can assume a substantially flattened configuration such that opposite internal surfaces of the one-piece member substantially contact one another; and
the at least one folded configuration is one wherein the member can assume a substantially flattened configuration when squeezed between a user's fingers of one hand.

16. A method of making the device of claim 1, the method comprising at least one of:
forming the member of an elastically expandable and/or foldable plastic material; and
forming the member having different diameter first and second sections.

17. A method of using the device of claim 1, the method comprising:
installing the member on a penis or a member simulating a penis; and
inserting the penis or member simulating the penis into a vagina in a manner which results in at least the first section providing stimulation to at least a portion of the vagina.

18. An enhancement device comprising:
an elastically expandable member having open opposite ends and being capable of assuming each of a generally cylindrical shape and at least one folded configuration;
a first section of the member having a first axial length;
a second section of the member having a second axial length and an outer diameter smaller than the first section; and
an overall length of the member, measured when the member is in a pre-installed and non-folded state, being at least 50% or more of an overall length of an erect penis or a member simulating a penis,
wherein the at least one folded configuration is one wherein the first section can be folded back at least partially over the second section during installation of the device.

19. A device for providing deep clitoral stimulation, comprising:
a member having open opposite ends;
the member capable of being folded upon itself;
a first section of the member having a first axial length;
a second section of the member having a second axial length and an outer diameter smaller than the first section; and
an overall length, measured when the member is in a pre-installed and non-folded state, that is at least 50% or more of an overall length of an erect penis or a member simulating a penis.

20. A device for providing deep clitoral stimulation, comprising:
a member having open opposite ends and an overall length measured when the member is in a pre-installed and non-folded state;
the member capable of being folded upon itself;
a first section of the member having a first length, a first wall thickness, and being configured to be closer to a base of the penis than to a head of the penis when the member is installed on the penis;
a second section of the member having a second length, a second wall thickness, and being configured to be closer to the head of the penis than to the base of the penis when the member is installed on the penis; and
the first wall thickness being greater than the second wall thickness,
wherein the overall length of the member is:
less than an overall length of the penis as measured between the base of the penis and a rear end of the head of the penis;
at least 50% or more than an overall length of the penis, and
wherein the penis is an erect penis or a member simulating a penis.
